(19) United States
(12) Patent Application Publication
(10) Pub. No.: US 2010/0136022 A1
(43) Pub. Date: Jun. 3, 2010

(54) METHOD FOR MODULATING THE IMMUNE RESPONSE BY ACTIVATING OR INACTIVATING THE NOTCH AND/OR STAT4 SIGNAL PATH

(75) Inventor: Alexander Scheffold, Berlin (DE)

Correspondence Address: JOYCE VON NATZMER;PEQUIGNOT + MYERS LLC, 200 Madison Avenue, Suite 1901, New York, NY, 10016, US

(73) Assignee: DEUTSCHES RHEUMA-FORSCHUNGSZENTRUM BERLIN, Berlin (DE)

(21) Appl. No.: 12/525,022

(22) PCT Filed: Jan. 30, 2008
(86) PCT No.: PCT/DE08/00210

§ 371 Date: Jan. 20, 2010

(30) Foreign Application Priority Data
Jan. 30, 2007 (EP) 07090012.1
Mar. 13, 2007 (EP) 07075192.0
May 30, 2007 (EP) 07090107.9

(51) Int. Cl. A61K 39/395; A61P 29/00

(52) U.S. Cl. 424/158.1; 424/172.1

(57) ABSTRACT

The invention relates to a method for modulating the inflammatory potential of T cells, in particular by regulating the IL-10 production in pro-inflammatory T cells, by influencing the activity or activation of the Notch and STAT4 signaling pathways. The invention also relates to the use of the method for inhibiting inflammations or in immunosuppression. More specifically, the invention relates to the use of the method in the treatment of diseases associated with inflammations. Furthermore, the invention relates to the use of the method for reducing the IL-10 production in pro-inflammatory T cells and thus the enhancement of T cell-mediated immune reaction, especially in the event of infections, tumor diseases or vaccinations against infections or tumors.

The invention relates to a method for the targeted modulation of anti-inflammatory functions of T cells by influencing the Notch and STAT4 signaling path-ways.

The invention comprises:
1. generation of anti-inflammatory T cells, especially T cells producing interleukin (IL) 10, by combined activation of the Notch signaling pathway and/or STAT4 signaling pathway. The invention also relates to the use of the method for inhibiting inflammations or in immunosuppression. More specifically, the invention relates to the use of the method for treating diseases associated with inflammations, and/or
2. generation of T cells with enhanced inflammatory function, especially by deactivating the IL-10 production in T cells by blocking the Notch and STAT4 signaling pathways. In this respect, the invention also relates to the use of the method to enhance the immune defense. More specifically, the invention relates to the use of the method for treating diseases associated with infections or tumors.

The immune system enables effective defense against pathogens as well as tolerance to autologous cells and harmless substances. The T helper cells are of crucial importance in maintaining this balance. Failure may give rise to autoimmune diseases or allergies.

In accordance with the diverse functions of T helper cells, there are different subpopulations of these cells. They are remarkable for their differential expression of soluble messengers, the cytokines.

One subpopulation of T helper cells, the so-called Th1 cells, express cytokines which are able to direct the cellular immune response, including interferon(IFN)-γ and tumor necrosis factors(TNF)-α and β. IFN-γ is responsible for the activation of macrophages and is necessary for the control of intracellular pathogens and viruses. Owing to their cytokines, Th1 cells have an inflammation-promoting effect and therefore are also referred to as pro-inflammatory.

Another subpopulation of T helper cells, the so-called Th17 cells, are characterized by the production of the IL-17 cytokine and likewise have a pro-inflammatory effect. In particular, Th17 cells seem to be important in the development of autoimmune diseases.

While this pro-inflammatory function is necessary for successful immune defense, chronic inflammations or autoimmune diseases may arise in the absence of appropriate counterregulation. The immune system utilizes various mechanisms to prevent uncontrolled activation of immune cells. One mechanism is release of immunosuppressive cytokines, of which above all IL-10 is of crucial importance.

IL-10 originally has been identified as a factor which inhibits cytokine secretion of Th1 cells and is produced by so-called Th2 cells. It is also produced by various populations of regulatory T cells (CD25+FoxP3+ and Tr1 cells). IL-10 has anti-inflammatory and suppressive effects on most hematopoietic cells. It indirectly inhibits the cytokine production and proliferation of antigen-specific effector T cells by inhibiting the antigen presentation by various APCs such as dendritic cells, Langerhans cells and macrophages.

Meanwhile, it is well-known that pro-inflammatory T cells themselves are able to produce IL-10 in order to limit inflammatory reactions triggered by them and prevent immunopathologies in this way. However, it has been unclear which type of signals could be used to regulate the IL-10 production in pro-inflammatory T cells. In particular, no method has been known for inducing or deactivating the IL-10 production in previously established pro-inflammatory Th1/Th17 cells, so-called memory cells.

Methods have been described wherein IL-10-producing T cells (Tr1 cells) can be generated from naive, i.e. antigen-inexperienced, T cells. Apart from IL-10, they produce no or few pro-inflammatory cytokines and therefore have an anti-inflammatory effect. In vitro culturing with immunosuppressive substances such as IL-10 itself, dexamethasone, vitamin D (inter alia) has been described. Furthermore, there have been reports according to which Notch signals alone can achieve formation of Tr1 cells. However, Notch can also induce Th1 or Th2 T cells. The precise molecular mechanisms and possibly necessary cofactors for these various differentiation pathways are unknown. More recently, however, it has been demonstrated for example that Notch signals via the Delta-like 4 ligand, together with interleukin 12, are necessary to induce strong pro-inflammatory Th1 response to viral infections. If and in which way Notch induces regulatory T cells is therefore unknown.

There have been reports that Notch signaling, generally in association with interferons and mainly type I interferons, gives rise to an increased IL-10 production and simultaneously reduces pro-inflammatory molecules. It has been assumed that treatment with interferon causes activation of STAT1 and STAT2 which mediate the intracellular effects of the interferon signal. Moreover, these reports fail to differentiate which ligands, Delta or Jagged family, could induce Notch effects.

Also, there have been reports that plasmacytoid dendritic cells (pDC) can induce IL-10 in T cells. It has been assumed that ICOS ligand or type I interferons, both of which being expressed at high levels by pDC after activation, are responsible for this.

In addition, these various methods are essentially characterized in that naive T cells or T cells not exactly defined (mixture of naive and memory T cells) are used as starting cells, which cells can be made to differentiate relatively easily by specific signals. In contrast, it is unknown how to achieve modulation in already differentiated memory T cells such as normally present in already established immunopathologies. Memory cells already have particular differentiation steps fixed therein, so that influencing memory T cells is generally more difficult to achieve, thereby impeding therapy of established inflammatory diseases.

The production of IL-10 by actually pro-inflammatory T cells can be regarded as a regulatory function which is to prevent excessive immune responses and immunopathologies resulting therefrom. On the other hand, however, IL-10 production can also prevent effective pathogen defense. In various infection models, for example, IL-10-producing Th1 cells are held responsible for inefficient elimination of pathogens and chronification of infections. The molecular signals which might be responsible for this have not been clearly defined, but the mechanisms described above might be relevant. Hence, by targeted and selective blocking of the IL-10 production it should be possible to enhance pathogen-targeted immune responses or improve vaccinations against pathogens or tumors.

The object of the invention was to provide a method that would allow inhibition or enhancement of inflammatory processes in an organism or in vitro by modulating the inflammation-mediating cells, especially by enhancing or deactivating the IL-10 production directly in inflammatory T cells. In particular, the intention was to provide a possible way of effecting a change in IL-10 production even in memory T cells and in T cells involved in conditions of chronic inflammation. In a particularly preferred fashion the method is intended to be used or applied independently of interferon.

The invention relates to the surprising teaching that it is possible to provide a method for modulating and generating anti-inflammatory functions in T cells, especially by modulating the IL-10 production in vitro or in vivo, preferably in the event of inflammatory reactions, wherein Notch and STAT4 signals are modulated, and wherein inhibition of the signals reduces the IL-10 production and enhances the inflammatory reaction, and activation of the signals enhances the IL-10 production and reduces the inflammatory reaction.

More surprisingly, the object of the invention can be accomplished by means of a method for generating modified T helper cells, wherein Th1 cells or Th17 cells are contacted with signal-active Notch molecules and signal-activated STAT4 molecules. As a result of combined activation of Notch and STAT4, simultaneously or time-shifted, the pro-inflammatory effect of Th1/17 is deactivated or reduced in that protective, particularly IL-10-producing, T cells are obtained. Accordingly, the method of the invention relates to the modulation of anti-inflammatory functions in T cells on the one hand and, on the other hand, to the generation of protective anti-inflammatory cells, particularly IL-10-producing cells, wherein T cells are contacted with signal-active Notch molecules and with signal-active STAT4 molecules so that the T cells are obtained and modulation of the anti-inflammatory function of the T cells is possible.

The invention also relates to a method for modulating anti-inflammatory functions in T cells, especially for deactivating IL-10 in these cells and generating highly reactive, inflammatory T cells, wherein contact of the T cells with signal-active Notch or STAT4 is prevented and the T cells are obtained in this way.

More specifically, it was surprising that activation of STAT4 (but not STAT1 or STAT2 or ICOS) with Notch was required for IL-10 induction because STAT4 is mainly held responsible for formation of pro-inflammatory Th1/Th17 cells. It was also surprising that Notch ligands of the Delta-like family, especially Delta-like 4, bring about IL-10 induction, while Jagged has no or rather even opposite effects.

The method according to the invention can preferably be used or applied interferon-independently.

It was also surprising that pDC, in particular following stimulation with ligands of the Toll-like receptors (TLR 1-13 and especially TLR9), express particularly large quantities of Delta-like 4 and under these conditions cause particularly efficient induction of IL-10 in T cells.

Furthermore, it was entirely surprising that an enhancement of the T cell reaction can be achieved by blocking the Notch signaling pathway, especially by reducing or deactivating the IL-10 production of the pro-inflammatory T cells.

The method according to the present application is remarkable for the following advantages:
Departure from conventional technologies
New field of problems
Existence of a long-unsatisfied, urgent need for the solution of the problem solved by the invention
Hitherto vain efforts in the art
Simplicity of a particular solution indicates inventive activity, especially as it replaces more complicated teachings
Development in scientific technology has proceeded in a different direction
Achievement that rationalizes development
Erroneous ideas in the art on the solution of the problem at issue (prejudice)
Technical progress, e.g. improvement, performance enhancement, lower expense, savings of time, materials, work steps, cost or raw materials difficult to obtain, enhanced reliability, elimination of flaws, superior quality, maintenance freedom, greater efficiency, higher yield, expansion of the technical scope, provision of a further means, creation of a second approach, creation of a new field, first-time solution of a problem, reserve means, alternatives, scope for rationalization, automation and miniaturization, or enrichment of the range of available drugs
Fortunate choice out of a variety of possibilities because one has been selected, the result of which has not been predictable, this therefore being a patentable fortunate choice
Errors in the technical literature or highly contradictory representation of the subject matter of the invention
Young field of technology
Combination invention, i.e., several known elements have been combined to achieve a surprising effect
Issue of licenses
Praise in the art
Economic success.

More specifically, the advantageous embodiments of the invention have at least one or more of the above-mentioned advantages.

Surprisingly, modification of naive and memory T helper cells by a combination of Notch and STAT4 can be used in a simple, safe and effective manner to restrict pro-inflammatory processes.

It was also surprising that, when contacted with signal-active Notch and signal-activated STAT4, pro-inflammatory Th1 and Th17 cells are converted into anti-inflammatory, particularly IL-10-producing, T cells.

Furthermore, it was surprising that blocking the Notch signaling pathway following immunization with antigen plus adjuvants (e.g. TLR ligands) can achieve a reduction in IL-10 production and an enhancement of the inflammatory T cell reaction.

A number of terms will be explained and illustrated below.

Modulation of Anti-Inflammatory Functions in T cells

In particular, this is understood to be influencing the expression of factors with an anti-inflammatory effect, especially IL-10, including anti-inflammatory soluble messenger substances and/or surface molecules. Modulation means that influencing may proceed in two different directions:
in the event of inflammatory diseases, enhancement of these factors would be desirable and could be achieved by activating the Notch signaling pathway;
in the event of (tumor) vaccination, a reduction of anti-inflammatory factors is preferred, which can be realized by inhibiting the Notch pathway.

Signal-Active Notch and STAT4 Molecules

For both Notch and STAT4 the statement holds that they are always present in a cell in latent form. This latent form is converted into an (signal) active (signal-active=active) form by activators. This proceeds by proteolytic cleavage in the event of the Notch receptor or by phosphorylation in the event of STAT4. At the same time, this means that the active form is different in structure/nature from the latent form.

Thus, a signal-active form can be conveyed into a cell on two routes:
a) By physiological activation of receptors: Activators of Notch according to this definition are Notch ligands or stimulating anti-Notch antibodies. In the event of STAT4 this concerns (IFN-independently) cytokines of the IL-12 family, IL-12, IL-23, IL-27 and IL-35.
b) By direct transfection of cells with the constitutively active form which, in the event of Notch, is the Notch intracellular domain (NICD) and in the event of STAT4 a modified STAT4 molecule which is phosphorylated in the cell independently of the receptor and thereby becomes active.

Highly Reactive Inflammatory T Cells

Highly reactive inflammatory T cells are inflammatory T cells wherein particularly the production of the anti-inflammatory cytokine IL-10 is inhibited (e.g. by blocking of Notch).

Notch Ligand Fragments and Protein Fragments

Notch ligand fragments and protein fragments are those as disclosed in WO 2004/024764 A1. The constructs and fragments of the Notch ligands disclosed therein can be used for either activation or inhibition of the Notch signaling pathway. In a preferred fashion the fragments have essentially the same activity as the molecule from which they have been obtained.

Substances Inhibiting the Natural Activation or Activity of STAT4

Inhibitors of STAT4 include, on the one hand, especially blocking antibodies against STAT4-activating cytokines (e.g. anti-IL-12, anti-IL-23, anti-IL-27, anti-IL-35) and, on the other hand, especially blocking antibodies against the corresponding cytokine receptors on the cells. More strictly speaking, STAT4 inhibitors could also be substances from the class of decoy oligonucleotides (nucleotides which include STAT4 binding sites and thus inhibit binding of active STAT4 to the target DANN in a cell) or proteins or fragments or derivatives thereof from the group of STAT-interacting proteins, such as SHIP, PIAS or SOCS. The latter are natural interaction partners of STAT, which can modulate the activity and are used as specific inhibitors after incorporation in a cell.

Pharmacological Inhibitors of Notch Activation

In particular, pharmacological inhibitors of the Notch signaling pathway comprise substances of the class of γ-secretase inhibitors.

In addition, other inhibitors of the Notch signaling pathway should be included, such as soluble Notch ligands, their fragments, fusion proteins, or single peptides such as disclosed in WO 2004/024764. More specifically, naturally blocking antibodies against Notch receptors are claimed as well. Inhibition of Notch activation is also possible by transfection of cells with negative regulators of the Notch signaling pathway. In this context, Deltex, MINT, NRARP or dominant-negative forms of Mastermind are preferred agents well-known to those skilled in the art.

The expression “modulation of the inflammatory potential” is equivalent to the expression “modulation of the (anti)inflammatory function”. In the meaning of the invention, enhancing or diminishing the anti-inflammatory T cell function (preferably IL-10 production) are alternative ways of manipulation by Notch, which can be used depending on the type of disease.

In a preferred embodiment of the invention, additional contacting of the Th1/Th17 cells with signal-active STAT4 molecules is envisaged. Combined activation of the Notch and STAT4 signaling pathways is necessary to achieve conversion of pro-inflammatory Th1/Th17 cells into anti-inflammatory cells with suppressant properties.

In another preferred embodiment the T cells in the methods according to the invention are selected from the group comprising naive T cells and/or memory cells. In a particularly preferred fashion the T cells are memory T cells.

In another preferred embodiment of the invention, selection of the T cells from the group of inflammatory Th1 and/or Th17 cells is envisaged.

As envisaged in another preferred embodiment of the invention, the Notch molecules in the meaning of the invention are selected from the group comprising Notch receptors or Notch ligands.

In another preferred embodiment of the invention the Notch receptors in the meaning of the invention are in particular molecules selected from the group comprising Notch1, 2, 3 and/or 4. The Notch ligands are in particular molecules selected from the group comprising molecules of the Jagged family, especially Jagged 1 and/or 2, and/or Delta-like (DII) family, especially DII1, 2, 3 and/or 4.

It is envisaged in another preferred embodiment of the invention that the ligands used in said method are molecules of the Delta-like family, especially Delta-like 4 and/or Delta-like 1.

Furthermore, it is envisaged in a preferred embodiment that said contacting with STAT4 molecules is effected by activation with cytokines, especially with the cytokines IL-12, IL-23 and/or IL-27, or by over expression of signal-active STAT4, especially by transduction of the T cells.

In another preferred embodiment of the invention, contacting with STAT4 molecules is effected by over expression of molecules which increase or improve the STAT4 activation, especially the cytokine receptors, preferably selected from the group comprising IL-12, IL-23 and/or IL-27 and/or fusion proteins of these cytokines with their receptor or STAT4 or signal-active STAT4, preferably by transduction of the T cells.

It is envisaged in a particularly preferred embodiment of the invention that contacting in the meaning of the invention is effected by over expression of signal-active Notch1, 2, 3 and/or 4 or by contacting endogenous Notch molecules with

Notch ligands. A person skilled in the art will be familiar with methods to induce over expression of particular proteins and produce signal-active variants of proteins. Furthermore, a person skilled in the art will be familiar with methods of contacting endogenous Notch molecules with Notch ligands in such a way that the Notch signaling pathway is activated.

In a particularly preferred embodiment of the invention, contacting with Notch ligands is effected by stimulation with antigen-presenting cells, said contacting being enhanced and/or induced especially via activation of Toll-like receptors (TLR) and/or CD40 on the antigen-presenting cells. A person skilled in the art will be familiar with methods to activate TLR. In a preferred fashion the ligands are molecules of the Delta-like family, especially Delta-like 4 and/or Delta-like 1. In particular, the TLRs are molecules selected from the group comprising TLR1, 2, 3, 4, 5, 6, 7, 8, 9, 10, 11, 12 and/or 13.

It is envisaged in another preferred embodiment of the invention that contacting with Notch ligands is effected by stimulation with plasmacytoid dendritic cells, said contacting particularly being enhanced and/or induced by stimulating the plasmacytoid dendritic cells via Toll-like receptors and/or CD40.

In a particularly preferred embodiment of the invention, contacting with Notch ligands is effected by stimulation with plasmacytoid dendritic cells, said contacting particularly being enhanced and/or induced via TLR. Plasmacytoid dendritic cells give particularly high levels of Delta-like 1 and 4 Notch ligand expression.

In another preferred embodiment of the invention, contacting with signal-active STAT4 molecules is effected by adding cytokines, especially the cytokines IL-12, IL-23, IL-27.

In another preferred embodiment of the invention, contacting with signal-active STAT4 molecules is effected by over expression of signal-active STAT4. A person skilled in the art will be familiar with methods to induce over expression of particular proteins and produce signal-active variants of proteins.

In another embodiment of the invention, generating the protective, especially the IL-10-producing T cells is preferably effected in vitro by stimulation with Notch ligand proteins or signal-active fragments of Notch ligands, by stimulation with Notch ligand-expressing cells and/or by incorporating signal-active Notch using viral and/or non-viral transduction methods in the presence of Th1/Th17 cells.

It is envisaged in another preferred embodiment of the invention that generating the protective, especially the IL-10-producing T cells is carried out in vivo by using recombinant Notch ligands and/or Notch ligand fusion proteins and/or signal-active Notch ligand fragments and/or by using Notch ligand-expressing cells. Among Notch ligand-expressing cells, plasmacytoid dendritic cells are particularly suitable.

Accordingly, the invention in a preferred embodiment relates to a method wherein generating the IL-10-producing T cells is effected in vitro by stimulation with Notch ligand proteins, Notch ligand fusion proteins and/or signal-active Notch ligand fragments, by stimulation with Notch ligand-expressing cells and/or by incorporating signal-active Notch using viral and/or non-viral transduction methods in the presence of signal-activated STAT4 molecules.

In another preferred embodiment the invention relates to a method wherein generating IL-10-producing T cells is effected in vivo by using recombinant Notch ligands and/or by using Notch ligand-expressing cells in the presence of signal-active STAT4.

In another preferred embodiment the invention relates to a method wherein inhibition of Notch and/or STAT4 activation is effected by antibodies against Notch or Notch ligands and/or against STAT4-activating cytokines, especially IL-12, IL-23, IL-27 and/or their receptors and/or by recombinant proteins or protein fragments or peptides of Notch or Notch ligands.

In another preferred embodiment the invention relates to a method wherein inhibition of STAT4 activation is effected by substances which inhibit the natural activation or activity of STAT4.

In another preferred embodiment the invention relates to a method wherein inhibition of Notch activation is effected by using pharmacological inhibitors of Notch activation, e.g. so-called γ-secretase inhibitors.

In another preferred embodiment the invention relates to a method wherein inhibition takes place in the course of an active immune reaction, especially during infections, tumor diseases, vaccinations.

In another preferred embodiment the invention relates to a method wherein inhibition proceeds in the course of a vaccination using antigens in particular of tumors or pathogens together with adjuvants, preferably ligands for Toll-like receptors (TLR), anti-CD40 and/or CD40 ligand.

In another preferred embodiment the invention relates to a method wherein the adjuvants are derived from the group of ligands for TLR9, preferably oligonucleotides comprising methylated CpG motifs.

In another preferred embodiment the invention relates to a method wherein the adjuvants are derived from the group of antibodies and/or ligands for CD40.

In another preferred embodiment the invention relates to a method wherein inhibition proceeds in the course of a vaccination with dendritic cells or other antigen-presenting cells, in particular also by using dendritic cells or other antigen-presenting cells for immunization, wherein expression of Notch ligands is inhibited (e.g. by transduction with siRNA or antisense oligonucleotides against the Notch ligands).

In another preferred embodiment the invention relates to a method wherein expression of Notch ligands of the Delta-like family, especially of the ligands Delta-like 1 and Delta-like 4, is inhibited in the dendritic cells or other antigen-presenting cells.

In another preferred aspect the invention relates to the use of the inventive method for inhibiting inflammations.

In another preferred aspect the invention relates to the use of the inventive method for immunosuppression.

In another preferred aspect the invention relates to the use of the inventive method for inducing interleukin 10 (IL-10).

In another preferred aspect the invention relates to the use of the inventive method for activating the immune response.

In another preferred aspect the invention relates to the use of the inventive method for enhancing vaccinations.

In another preferred aspect the invention relates to the use of the inventive method for inhibiting the interleukin 10 (IL-10) expression.

In another preferred aspect the invention relates to the use of the inventive method in the production of a medicament for inducing interleukin 10 (IL-10) for the immunosuppression in organ transplantations.

In another preferred embodiment of the invention it is envisaged to inhibit the combined activation of Notch and STAT4 in T cells so as to achieve an enhancement of the immune reaction by reducing the IL-10 production. This can be achieved by pharmacological inhibition of the Notch signal (a person skilled in the art will be familiar with methods of inhibiting the Notch activation using pharmacological means, e.g. by means of the γ-secretase inhibitor class of active substances) or by administering antibodies against Notch ligands (especially the Delta-like family and in particular against Delta-like 4) or by means of parts of ligands or peptides or other molecules blocking the signal-activating interaction between Notch and Notch ligands. Furthermore, this effect can be achieved by inhibiting the STAT4 activation. This can be done by using antibodies against STAT4-activating cytokines (IL-12, IL-23, IL-27, type I interferons) or receptors thereof or pharmacological substances blocking the STAT4 activation or function.

In another aspect the invention relates to the use of the inventive method for inhibiting inflammations, for immunosuppression and/or induction of IL-10.

In another preferred aspect the invention relates to the use of the inventive method for activating pro-inflammatory T cell functions, for vaccinations in the event of infections or tumor diseases and/or inhibiting the IL-10 production.

The invention also relates to the use of the inventive method in the production of a medicament to inhibit inflammations, especially for the treatment of diseases selected from the group comprising HASHIMOTO thyroiditis, primary myxedema, thyrotoxicosis (BASEDOW disease), pernicious anemia, ADDISON disease, myasthenia gravis, juvenile diabetes mellitus, GOODPASTURE syndrome, autoimmune hemolytic anemia, autoimmune leukopenia, pemphigus vulgaris, sympathetic ophthalmia, primary biliary cirrhosis, in particular primary biliary liver cirrhosis, autoimmune hepatitis, in particular chronic aggressive autoimmune hepatitis, SJOGREN syndrome, rheumatoid arthritis, rheumatic fever, systemic lupus erythematosus, dermatomyositis/polymyositis, progressive systemic sclerosis, WEGENER granulomatosis, panarteritis nodosa and/or hypersensitivity angiitis, thyrotoxicosis, thyroid-caused myxedema, generalized endocrinopathy, chronic gastritis type A, autoimmune diseases of single or all corpuscular elements of the blood, particularly idiopathic thrombocytopenia or thrombocytopathy, idiopathic leukopenia, agranulocytosis, pemphigoid, uveitis, diabetes mellitus type I, CROHN disease, ulcerative colitis, ADDISON disease, lupus erythematosus disseminatus and discoid form of said disease, as dermatomyositis and scleroderma, rheumatoid arthritis (=primary chronic polyarthritis), antiglomerular basement membrane nephritis, an aggressive immune reaction due to breakdown of the immune tolerance to self-determinants and a reduction of the activity of T suppressor cells, preferably with lymphocyte marker T8, or an excess of T helper cells, preferably with lymphocyte marker T4, over the suppressor cells; formation of autoantigens, particularly by coupling of host proteins to haptens, preferably drugs, immune reactions caused by ontogenetic tissue not developing until self-tolerance has developed, by protein components demasked as a result of conformational changes of proteins, preferably in connection with infections by viruses or bacteria and/or by new proteins formed in association with neoplasias.

In another preferred embodiment the invention also relates to the use of the inventive method in the production of a medicament for immunosuppression for the treatment of diseases from the group comprising AIDS, acne, albuminuria (proteinuria), alcohol withdrawal syndrome, allergies, alopecia (loss of hair), ALS (amyotrophic lateral sclerosis), Alzheimer's disease, senile macular retinal degeneration, anemia, anxiety syndrome, anthrax (milzbrand), aortic sclerosis, occlusive arterial disease, arteriosclerosis, arterial occlusion, arteriitis temporalis, arteriovenous fistula, arthritis, arthrosis, asthma, respiratory insufficiency, autoimmune disease, atrioventricular block, acidosis, prolapsed intervertebral disc, inflammation of the peritoneum, pancreatic cancer, Becker muscular dystrophy, benign prostate hyperplasia (BPH), bladder carcinoma, hemophilia, bronchial carcinoma, breast cancer, BSE, Budd-Chiari syndrome, bulimia nervosa, bursitis, Byler syndrome, bypass, chlamydia infection, chronic pain, cirrhosis, commotio cerebri (brain concussion), Creutzfeld-Jacob disease, intestinal carcinoma, intestinal tuberculosis, depression, diabetes insipidus, diabetes mellitus, diabetes mellitus juvenilis, diabetic retinopathy, Duchenne muscular dystrophia, duodenal carcinoma, dystrophia musculorum progressiva, dystrophia, ebola, eczema, erectile dysfunction, obesity, fibrosis, cervix cancer, uterine cancer, cerebral hemorrhage, encephalitis, loss of hair, hemiplegia, hemolytic anemia, hemophilia, pet allergy (animal hair allergy), skin cancer, herpes zoster, cardiac infarction, cardiac insufficiency, cardiovalvulitis, cerebral metastases, cerebral stroke, cerebral tumor, testicle cancer, ischemia, Kahler's disease (plasmocytoma), polio (poliomyelitis), rarefaction of bone, contact eczema, palsy, liver cirrhosis, leukemia, pulmonary fibrosis, lung cancer, pulmonary edema, lymph node cancer, (Morbus Hodgkin), lymphogranulomatosis, lymphoma, lyssa, gastric carcinoma, mammary carcinoma, meningitis, milzbrand, mucoviscidosis (cystic fibrosis), multiple sclerosis (MS), myocardial infarction, neurodermitis, neurofibromatosis, neuronal tumors, kidney cancer (kidney cell carcinoma), osteoporosis, pancreas carcinoma, pneumonia, polyneuropathies, potency disorders, progressive systemic sclerosis (PSS), prostate cancer, urticaria, traumatic paraplegic syndrome, rectum carcinoma, pleurisy, craniocerebral trauma, vaginal carcinoma, sinusitis, esophagus cancer, tremor, tuberculosis, tumor pain, vaginal carcinoma, burns/scalds, intoxications, viral meningitis, menopause, soft-tissue sarcoma, soft-tissue tumor, cerebral blood circulation disorders and/or CNS tumors.

In another preferred embodiment the invention also relates to the use of the inventive method in the production of a medicament for immunosuppression in the event of organ transplantations.

In another preferred embodiment the invention relates to the use of the inventive method for inducing interleukin 10 in vitro or in vivo, especially in a patient, for the treatment of the above-mentioned diseases.

Without intending to be limiting, the invention will be explained in more detail with reference to the examples.

### EXAMPLE 1

Naive, i.e. antigen-inexperienced, CD4+CD25-CD62L+T helper cells were isolated using flow cytometry. The cells were subsequently cultured with irradiated (30 Gy) MHCII+ antigen-presenting cells at a ratio of 1:4 in the presence of interleukin 12 (10 ng/ml). Stimulation was effected polyclonally using 0.5 μg/ml anti-CD3 and 1 μg/ml anti-CD28. After 24 h the cells were retrovirally transduced with active Notch3 (Notch3IC). To this end, the cells were centrifuged with virus supernatant for 75 min at 750×g and 32° C. After 5 days the cells were restimulated with PMA/ionomycin, and their cytokine expression was analyzed using intracellular staining.

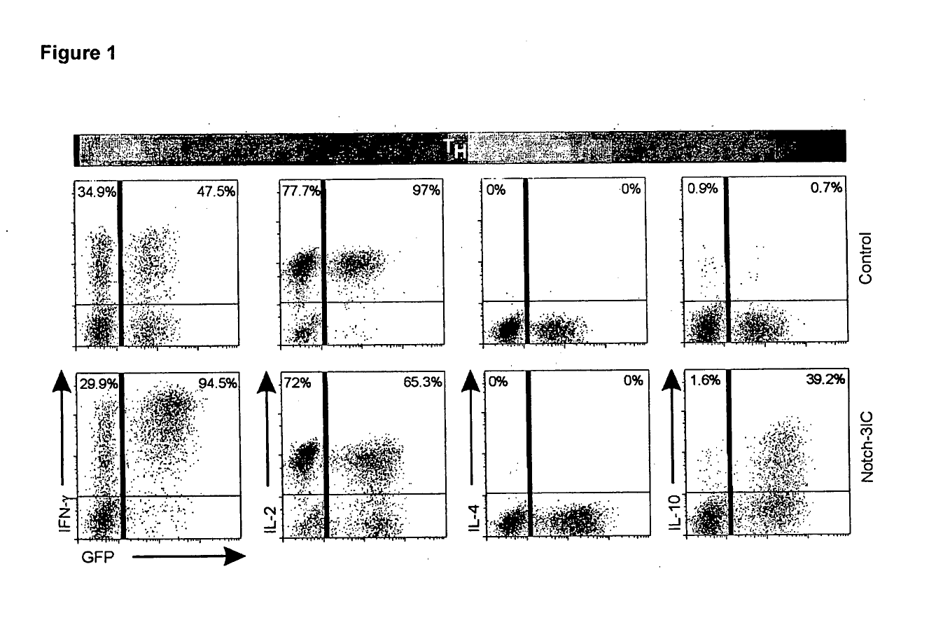

As illustrated in FIG. 1, control cells produce IFN-γ and IL-2 under these conditions, but no immunosuppressive IL-10. In contrast, Notch-transduced T helper cells produce less IL-2, but large quantities of IL-10 and IFN-γ. No IL-4 expression is observed.

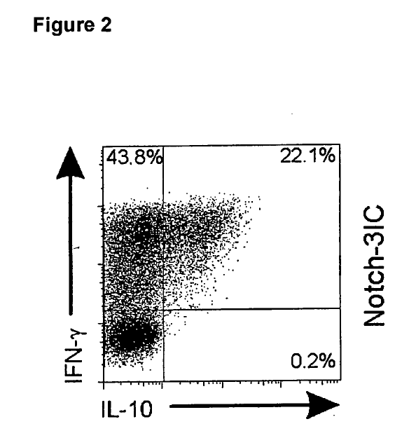

As seen in FIG. 2, Notch-modified Th1 cells simultaneously express the cytokines IFN-γ and IL-10.

### EXAMPLE 2

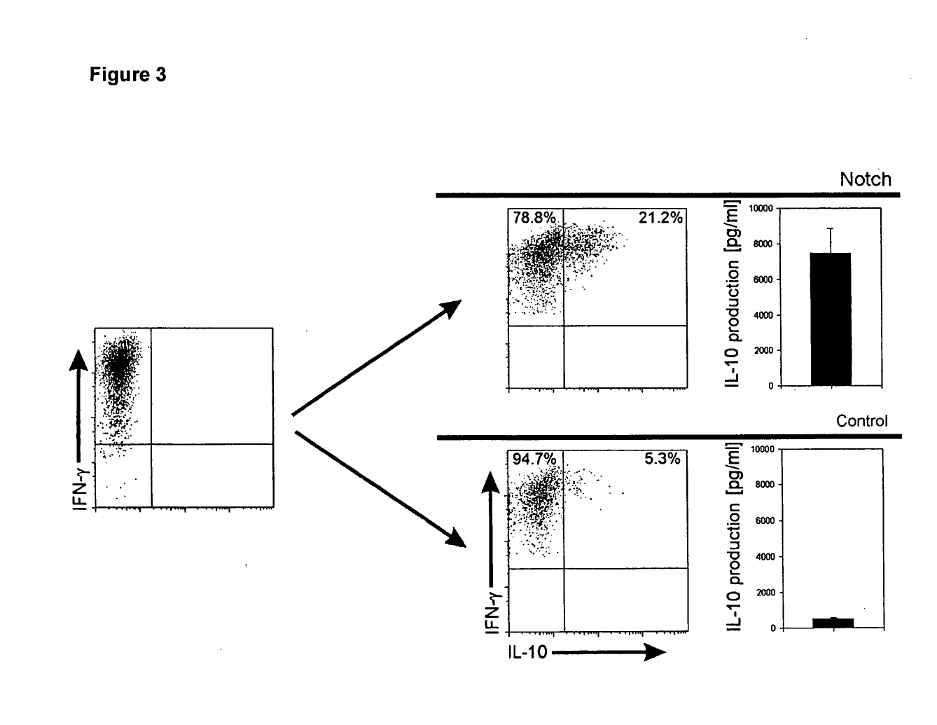

Naive CD4+CD25-CD62L+Th cells were isolated using flow cytometry and cultured for one week under Th1 conditions (10 ng/ml IL-12, 10 μg/ml anti-IL4, 0.5 μg/ml anti-CD3, 1 μg/ml anti-CD28). Thereafter, IFN-γ-producing cells were isolated by flow cytometry using the Miltenyi IFN-γ secretion assay. The cells were recultured with irradiated MHCII+ antigen-presenting cells under Th1 conditions. After 24 h the cells were retrovirally transduced with active Notch. After another 5 days the cells were restimulated with PMA/ionomycin, and their cytokine expression was analyzed. As illustrated in FIG. 3, control cells produce IFN-γ under these conditions, but no IL-10. In contrast, Notch-transduced T hel-per cells produce large quantities of IL-10 and IFN-γ. The differences in IL-10 production become particularly evident when measuring the cytokine in the culture supernatant (using ELISA).

### EXAMPLE 3

Naive CD4+CD25-CD62L+T helper cells from wild-type (C57BU6) or STAT4-deficient mice were isolated using flow cytometry. The cells were subsequently cultured with irradiated (30 Gy) MHCII+ antigen-presenting cells at a ratio of 1:4 in the presence of interleukin 12 (10 ng/ml). Stimulation was effected polyclonally using 0.5 μg/ml anti-CD3 and 1 μg/ml anti-CD28. After 24 h the cells were retro-virally transduced with active Notch3 (Notch3IC). To this end, the cells were centrifuged with virus supernatant for 75 min at 750×g and 32° C. After 5 days the cells were restimulated with PMA/ionomycin, and their cytokine expression was analyzed using ELISA.

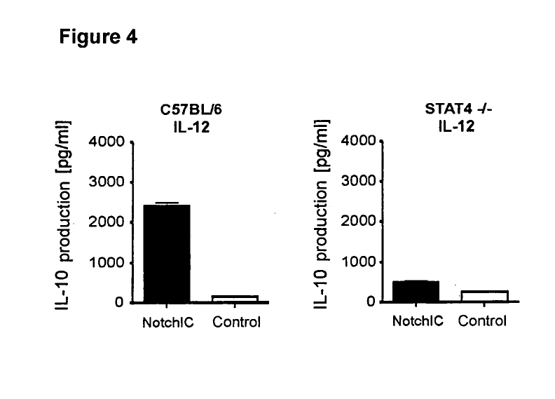

As illustrated in FIG. 4, Notch-transduced Th cells from wild-type mice produce large quantities of IL-10. No IL-10 production is observed in the event of STAT4-deficient cells. As a consequence, activation of the Notch and STAT4 signaling pathways to induce IL-10 is advantageous.

### EXAMPLE 4

Naive CD4+CD25-CD62L+Th cells were isolated using flow cytometry and cultured under Th1 conditions (10 ng/ml IL-12, 10 μg/ml anti-IL4, 0.5 μg/ml anti-CD3, 1 μg/ml anti-CD28). After 24 h the cells were retrovirally transduced with active forms of different Notch isotypes (Notch1 to 4). To this end, the cells were centrifuged with virus supernatant for 75 min at 750×g and 32° C. After 5 days the cells were restimulated with PMA/ionomycin, and their cytokine expression was analyzed using intracellular staining.

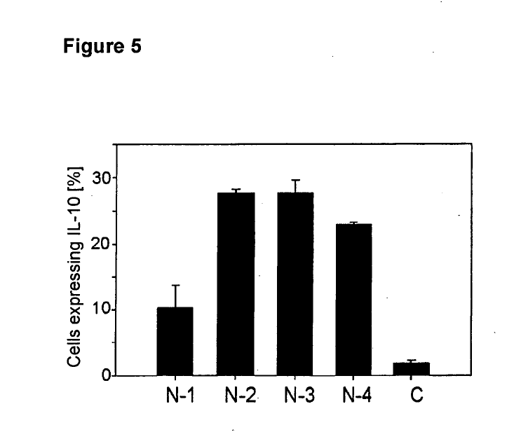

As seen in FIG. 5, IL-10 induction is observed in each case. Consequently, all four Notch isoforms can equally furnish IL-10 induction.

### EXAMPLE 5

Naive CD4+-CD25-CD62L+T helper cells from wild-type (C57BU6) or STAT4-deficient mice were isolated using flow cytometry. The cells were subsequently cultured with irradiated (30 Gy) MHCII+ antigen-presenting cells at a ratio of 1:4 in the presence of interleukin 27 (10 ng/ml). Stimulation was effected polyclonally using 0.5 μg/ml anti-CD3 and 1 μg/ml anti-CD28. After 24 h the cells were retro-virally transduced with active Notch3 (Notch3IC). To this end, the cells were centrifuged with virus supernatant for 75 min at 750×g and 32° C. After 5 days the cells were restimulated with PMA/ionomycin, and their cytokine production was analyzed using ELISA.

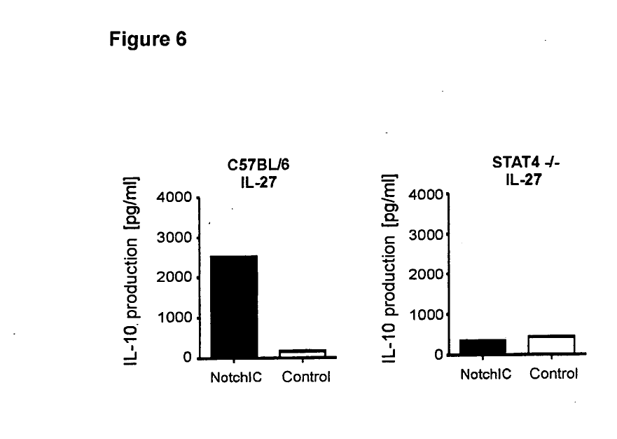

As illustrated in FIG. 6, Notch-transduced Th cells from wild-type mice produce large quantities of IL-10. No IL-10 production is observed in the event of STAT4-deficient cells. STAT4-dependent and together with Notch, administration of IL-27 consequently results in induction of IL-10 in the same way as of IL-12.

### EXAMPLE 6

Naive CD4+CD25-CD62L+Th cells were isolated using flow cytometry and cultured under Th1 conditions (10 ng/ml IL-12, 10 μg/ml anti-IL4) in the absence of antigen-presenting cells. Stimulation was effected using immobilized anti-CD3 (3 μg/ml) and 1 μg/ml anti-CD28. After 24 h the cells were retrovirally transduced with active Notch3 (Notch3IC). To this end, the cells were centrifuged with virus supernatant for 75 min at 750×g and 32° C. After 5 days the cells were restimulated with PMA/ionomycin, and their cytokine expression was analyzed using intracellular staining.

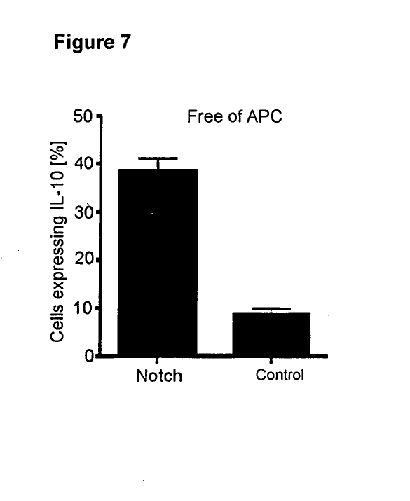

As illustrated in FIG. 7, Notch induces the immunosuppressive cytokine IL-10 even in the absence of antigen-presenting cells under Th1 conditions. Consequently, it can be concluded that no further costimulatory signals are required for this purpose.

### EXAMPLE 7

Naive CD4+CD25-CD62L+T helper cells were isolated by means of flow cytometry. The cells were subsequently cultured with irradiated (30 Gy) MHCII+ antigen-presenting cells at a ratio of 1:4 in the presence of interleukin 12 (10 ng/ml). Stimulation was effected polyclonally using 0.5 μg/ml anti-CD3 and 1 μ/ml anti-CD28. After 24 h the cells were retrovirally transduced with active Notch3 (Notch3IC). To this end, the cells were centrifuged with virus supernatant for 75 min at 750×g and 32° C. After 5 days, GFP+, i.e. Notch or control-transduced cells, were isolated using flow cytometry. Thereafter, naive CD4+CD25-CD62L+Thy1.1+ congenic Th cells and CD11c+ dendritic cells were isolated from spleen and lymphatic nodes of naive mice. The naive T cells were labeled with the CFDA-SE proliferation marker and cultured with the Notch or control-transduced cells at a ratio of 2:1 together with dendritic cells for 3 days (ratio of total T cells to dendritic cells 35:1). Subsequently, the proliferation of the naive T cells was analyzed using flow cytometry.

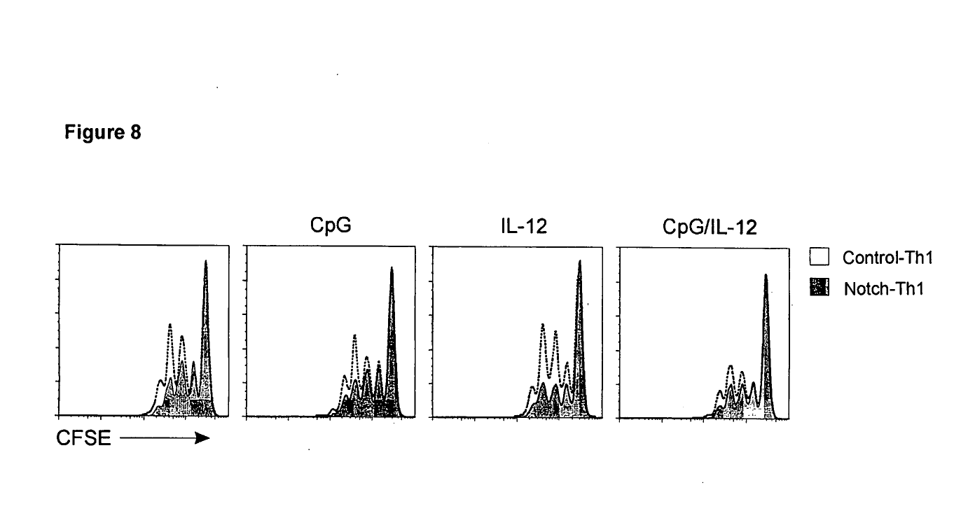

As seen in FIG. 8, Notch-transduced cells suppress proliferation of the naive Th cells. This suppression mediated by the notch-modified Th1 cells is particularly pronounced when IL-12 is added (10 ng/ml).

### EXAMPLE 8

Antigen-specific (OVA-TZR transgenic) naive CD4+CD25-CD62L+T helper cells were isolated using flow cytometry. The cells were subsequently cultured with irradiated (30 Gy) MHCII+ antigen-presenting cells at a ratio of 1:4 in the presence of interleukin 12 (10 ng/ml). Stimulation was effected using 0.2 μg/ml OVA peptide. After 24 h the cells were retrovirally transduced with active Notch3 (Notch3IC). To this end, the cells were centrifuged with virus supernatant for 75 min at 750×g and 32° C. After 5 days, GFP+, i.e. Notch- or control-transduced cells, were isolated by means of flow cytometry.

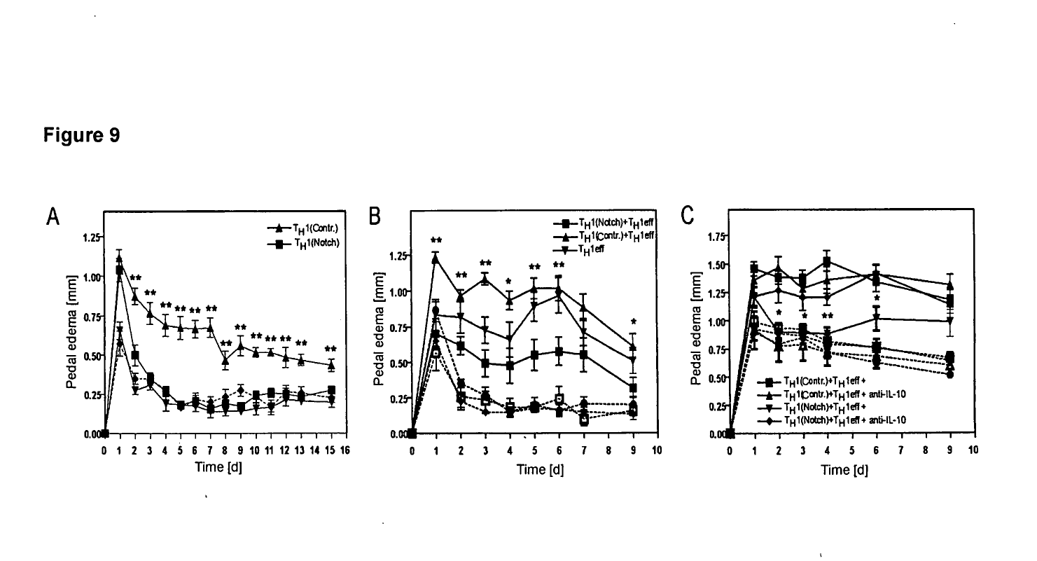

When transferring antigen-specific Notch-transduced Th1 cells in recipient animals and subsequently immunizing the animals with the antigen (OVA peptide in IFA), there is no inflammatory reaction (DTH) such as in the event of transferred control Th1 cells (FIG. 9A). Notch-modified Th1 cells lose their pro-inflammatory potential. Moreover, when transferred together with control Th1 cells, they are able to suppress the inflammatory reaction caused by the control cells, i.e., they can have a protective effect (FIG. 9B). When blocking IL-10 in the recipient animals, the suppression mediated by the Notch-transduced Th1 cells is terminated (FIG. 9C).

### EXAMPLE 9

Antigen-specific (OVA-TZR transgenic) naive CD4+CD25-CD62L+T helper cells were isolated using flow cytometry, cultured with A20-B cells (at a ratio of 2:1) in the presence of IL-12 (10 ng/ml), which cells express the Notch ligands DII-1 or DII-4, and stimulated with OVA peptide (0.2 μg/ml). After 5 days the cells were restimulated with PMA/ionomycin, and their cytokine expression was analyzed using intracellular staining.

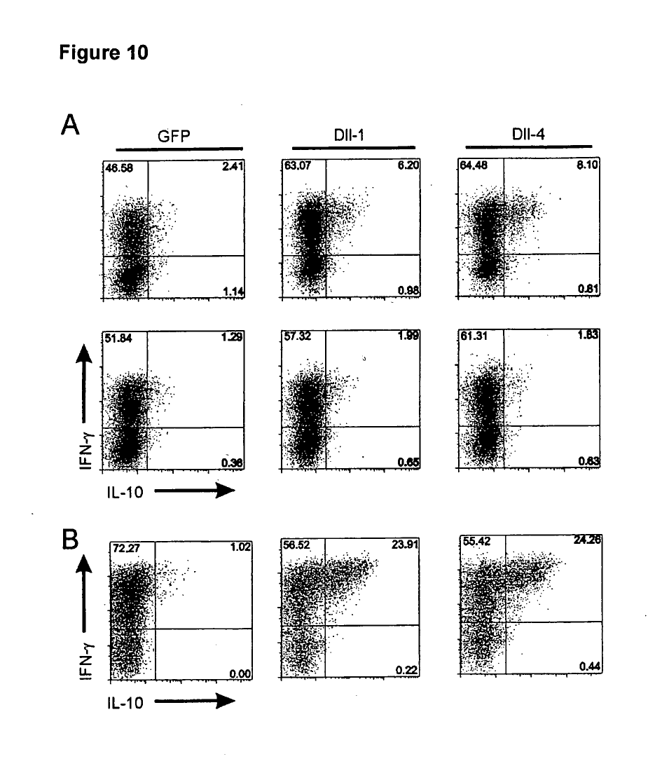

As illustrated in FIG. 10, coculturing with Delta-like ligands induces IL-10 production in the T cells. This induction can be blocked in a specific manner by an inhibitor of the Notch signaling pathway (γ-secretase inhibitor, 125 nM Insolution γ-secretase inhibitor X, Calbiochem) (FIG. 10A). Further coculturing in the presence of the DII-1 and DII-4 ligands dramatically increases the IL-10 production (FIG. 10B).

### EXAMPLE 10

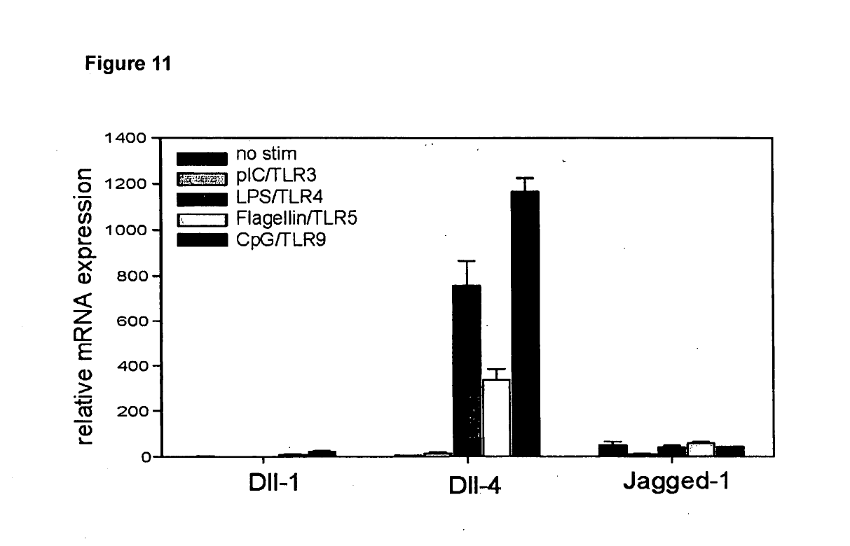

CD11c-positive dendritic cells were isolated from spleen and lymphatic nodes of naive mice using flow cytometry and stimulated with 50 μg/ml pIC (TLR3), 1 μg/ml LPS (TLR4), 1 μg/ml flagellin (TLR5) or 1 μM CpG (TLR9) for 15 h. As shown in FIG. 11, this results in enhanced expression of the Delta-like 4 Notch ligands. Expression of Delta-like 1 and Jagged 1 remains largely unchanged.

### EXAMPLE 11

Antigen-specific (OVA-TZR transgenic) naive CD4+CD25-CD62L+T helper cells were isolated using flow cytometry, cultured with CD11c+ dendritic cells from spleen and lymphatic nodes (at a ratio of 20:1) in the presence of IL-12 (10 ng/ml) and/or 1 μM CpG and stimulated with OVA peptide (0.2 μg/ml). After 5 days the cells were restimulated with PMA/ionomycin, and their cytokine expression was analyzed using intracellular staining.

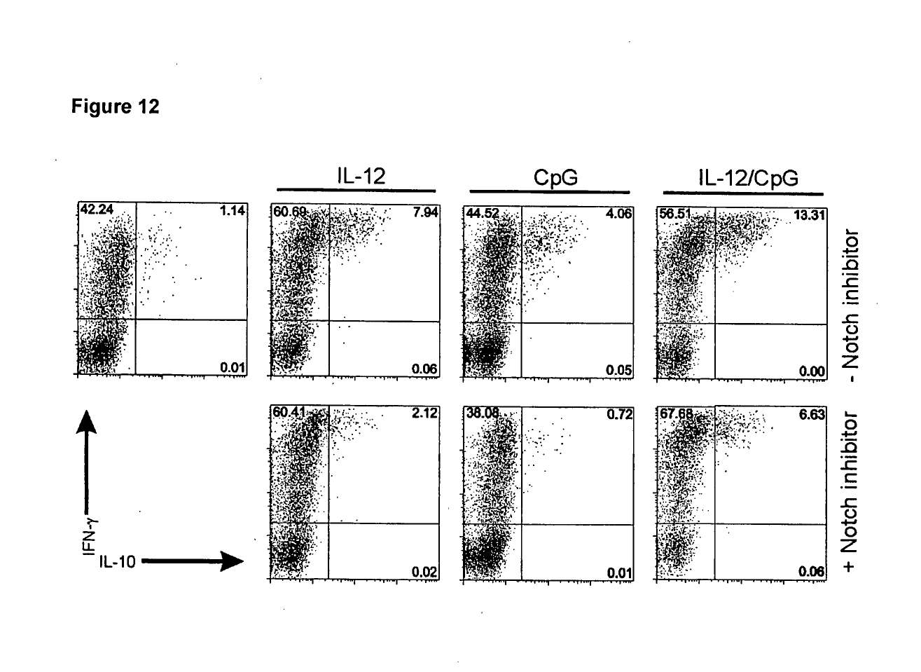

As shown in FIG. 12, IL-10 is induced in the T cells under these conditions. The expression of IL-10 can be blocked by a specific inhibitor of the Notch signaling pathway (γ-secretase inhibitor, Calbiochem, 125 nM).

### EXAMPLE 12

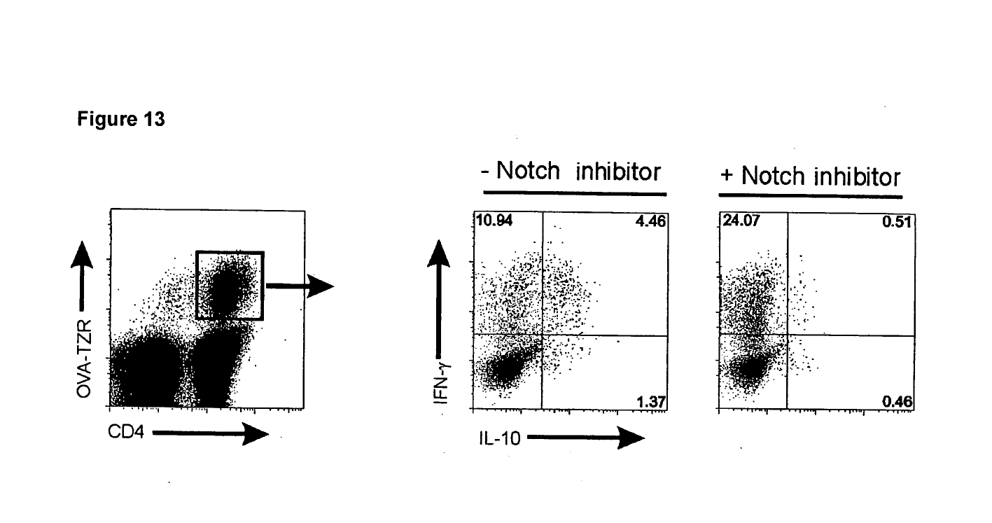

1·10⁷ antigen specific (OVA-TZR transgenic) naive Th cells were transferred into recipient mice which were subsequently immunized s.c. with 50 μg of OVA/20 μg of CpG. After 5 days the lymphatic node cells were restimulated with PMA/ionomycin and analyzed using intracellular cytokine staining. IFN-γ/IL-10 double-positive cells were induced under these conditions. The expression of IL-10 was blocked in mice which had received 10 μM/kg of a Notch inhibitor (γ-secretase inhibitor, DBZ, Syncom) (FIG. 13).

### EXAMPLE 13

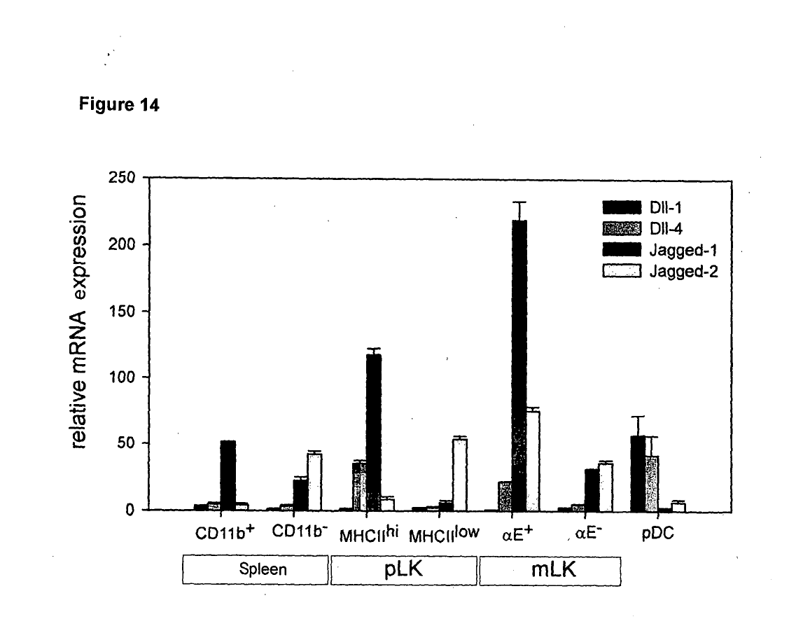

Various subpopulations of dendritic cells were isolated from spleen and lymphatic nodes using flow cytometry and compared for expression of Notch ligands. As shown in FIG. 14, it is only plasmacytoid dendritic cells that express the Delta-like 1 and Delta-like 4 ligands. Jagged ligands are absent here, while all other investigated types of DC express Jagged but not Delta-like.

### EXAMPLE 14

Antigen-specific (OVA-TZR transgenic) naive CD4+CD25-CD62L+T helper cells were isolated using flow cytometry, cultured with various subtypes of dendritic cells from spleen and lymphatic nodes, likewise isolated by means of flow cytometry, at a ratio of 20:1 in the presence of IL-12 (10 ng/ml) and stimulated with OVA peptide (0.2 μg/ml). Restimulation with PMA/ionomycin and analysis of the IL-10 production were performed after 5 days by means of ELISA.

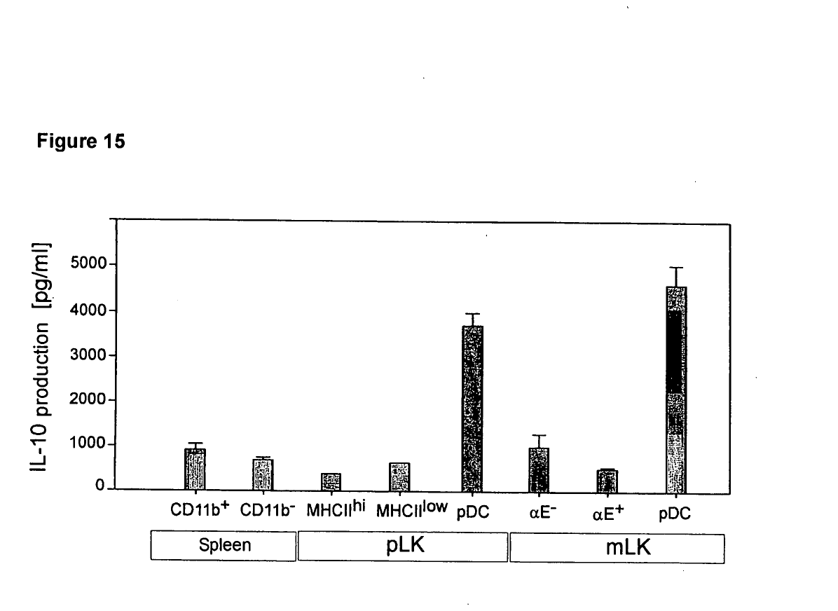

As shown in FIG. 15, it is primarily plasmacytoid dendritic cells that induce major quantities of IL-10 under these conditions.

### EXAMPLE 15

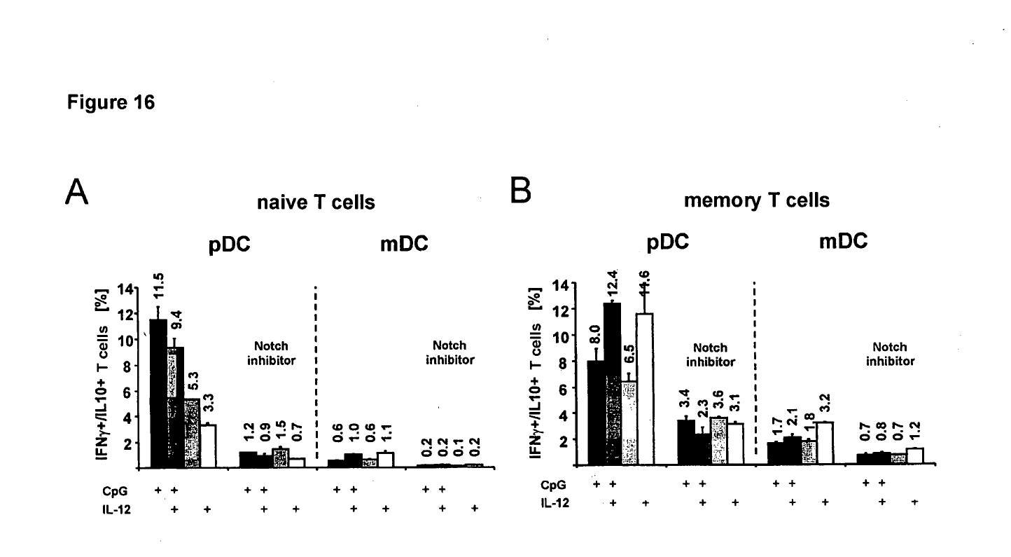

Human naive CD4+CD45RO-CD45RA+ and CD4+CD45RO+CD45RA memory T cells as well as plasmacytoid (BDCA4+) and myeloid (CD19-CD1c+) dendritic cells were isolated from whole blood using flow cytometry. The T cells were co-cultured with the dendritic cells at a ratio of 15:1 and stimulated with 1 μg/ml SEB each time. A number of batches were added with 3 μg/ml CpG, 10 ng/ml IL-12 or 85 nM γ-secretase inhibitor (Insolution γ-secretase inhibitor X, Calbio-chem). After 6 days the cells were restimulated with PMA/ionomycin, and their cytokine expression was analyzed using intracellular staining. IL-10 production is observed after coculturing with plasmacytoid, but not with myeloid dendritic cells. IL-10 expression is enhanced by CpG and can be blocked by inhibiting the Notch signaling pathway (FIG. 16A). After coculturing with plasmacytoid dendritic cells, memory T cells show a stronger IL-10 production compared to that with myeloid dendritic cells, which can be blocked in either case by inhibiting the Notch signaling pathway (FIG. 16B).

### EXAMPLE 16

Human CD4+ T cells were stimulated without antigen-presenting cells using immobilized anti-CD3 (3 μg/ml), anti-CD28 (1 μg/ml) and 10 ng/ml IL-12 and after 36 h retrovirally transduced with active Notch. To this end, the cells were centrifuged with virus supernatant for 75 min at 750×g and 32° C. After 6 days the cells were restimulated with PMA/ionomycin, and cytokine production was analyzed using intracellular staining.

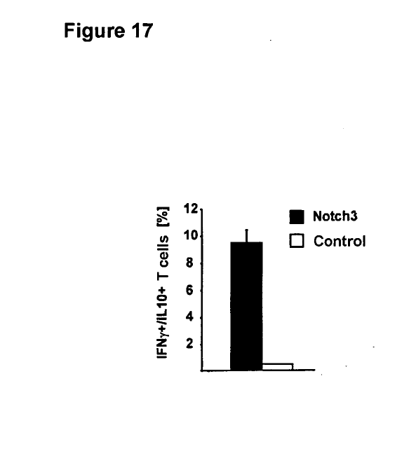

As illustrated in FIG. 17, induction of IL-10 takes place after Notch transduction.

### EXAMPLE 17

Isolated naive CD4+CD45RB+Th cells or Notch- or control-transduced Th1 cells of T lymphocytes were transferred into SCID mice. To investigate the suppressive potential of the notch-modified cells, isolated naive CD4+CD45RB+Th cells were cotransferred with Notch- or control-transduced Th1 cells. The health status of the animals was regularly checked by weighing. Initial signs of colitis were detected after about 3. weeks: weight loss, shaggy fur and soft stool. To analyze the course of the disease, the average weight change was plotted versus the initial value. In addition, the number of diseased animals per test group was determined. To this end, a weight loss of more than 5% relative to the initial value was interpreted as sign of a disease.

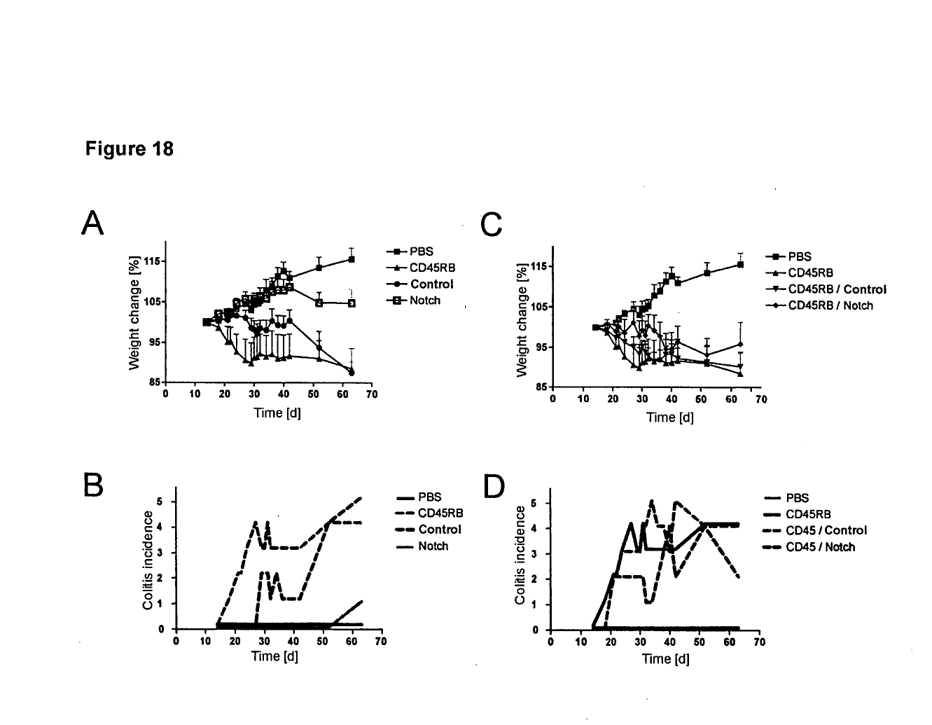

As shown in FIG. 18A, animals which had no cell transfer (PBS) exhibited continuous weight gain over the entire period of analysis. Between day 15 and day 30, animals which had received naive T cells only exhibited weight loss. Up to day 40 the weight of animals which had received control Th1 cells remained unchanged on an average. Thereafter, they exhibited weight loss as well. In contrast, animals which had received the Notch Th1 cells exhibited continuous weight gain up to day 40. This corresponded precisely to the control group without cell transfer. When considering the number of diseased animals per test group for assessment, the following scenario was observed: 0/6 animals were diseased in the PBS control group as expected, 4/6 animals in the control group with transfer of naive cells were diseased after about 30 days. At the same point in time it was 2/5 animals in the group which had received control Th1 cells. This number rose to 5/5 by day 60. As for the animals which had received the notch-modified Th1 cells, it was not until day 52 that 1/6 animals were diseased (FIG. 18B).

Animals which had received a cotransfer of control-transduced T cells showed a similar weight loss as the control group with transfer of naive T cells alone. The number of diseased animals per group was also comparable. From day 25 on, it was 4/6 in the control group on an average. In the group with cotransfer of control Th1 cells it varied between 3/6 and even 5/6. After cotransfer of notch-modified Th1 cells, a milder course of the disease was seen. The average weight in this group was 100% up to about day 35. Later on a decline to an average of 95% occurred. Hence, the onset of the disease was delayed by 10-15 days and there was an attenuation during the further course compared to the control group. With some fluctuations, the incidence shows a similar scenario: up to day 35 it was 1/6 to 2/6, compared to 3/6 to 4/6 after control cotransfer. During the further course the incidence in the Notch cotransfer group varied strongly between 2/6 and 4/6, so that it was either clearly below that of the control group or equivalent thereto at other points in time (FIG. 18C/D).

1. A method for modulating anti-inflammatory functions in T cells and generating protective, anti-inflammatory, in particular IL-10-producing T cells,
wherein
Notch signals and STAT4 signals are modulated, and wherein inhibition of the signals reduces the IL-10 production and enhances the an inflammatory reaction and activation of the signals enhances the IL-10 production and reduces the inflammatory reaction.

2. The method for modulating anti-inflammatory functions in T cells and generating protective, anti-inflammatory, in particular IL-10-producing T cells according to claim 1, wherein T cells are contacted with signal-active Notch molecules and with signal-active STAT4 molecules to obtain the T cells.

3. The method according to claim 1 for modulating anti-inflammatory functions in T cells, especially for deactivating IL-10 in these cells, and generating highly reactive, inflammatory T cells, wherein contact of the T cells with signal-active Notch or STAT4 is prevented to obtain the T cells.

4. The method according to claim 1,
wherein
antigen-presenting cells, particularly via stimulation of the antigen-presenting cells with Toll-like receptors and/or CD40.

14. The method according to claim 12,
wherein
contacting with Notch ligands is effected by stimulation with plasmacytoid dendritic cells, said contacting being enhanced and/or induced in particular by stimulating the plasmacytoid dendritic cells via Toll-like receptors and/or CD40.

15. The method according to claim 1,
wherein
generating the IL-10-producing T cells is effected in vitro by stimulation with Notch ligand protein, Notch ligand fusion proteins and/or signal-active Notch ligand fragments, by stimulation with Notch ligand-expressing cells and/or by incorporating signal-active Notch using viral and/or non-viral transduction methods in the presence of signal-activated STAT4 molecules.

16. The method according to claim 1,
wherein
generating IL-10-producing T cells is effected in vivo by using recombinant Notch ligands and/or by using Notch ligand-expressing cells in the presence of signal-active STAT4.

17. The method according to claim 1,
wherein
inhibition of Notch and/or STAT4 activation is effected by antibodies against the ligands are molecules of the Delta-like family, in particular Delta-like 4 and/or Delta-like 1.

10. The method according to claim 2,
wherein
contacting with STAT4 molecules is effected by activation with cytokines, in particular with cytokines IL-12, IL-23 and/or IL-27, or by over expression of signal-active STAT4, especially by transduction of the T cells.

11. The method according to claim 2,
wherein
contacting with STAT4 molecules is effected by over expression of molecules which increase or improve STAT4 activation, especially the cytokine receptors, preferably IL-12, IL-23 and/or IL-27 and/or fusion proteins of these cytokines with their receptor or STAT4 or signal-active STAT4, preferably by transduction of the T cells.

12. The method according to claim 2,
wherein
contacting is effected by over expression of signal-active Notch1, 2, 3 and/or 4, by contacting with endogenous Notch molecules and/or by contacting with Notch ligands.

13. The method according to claim 12,
wherein
contacting with the Notch ligands is enhanced and/or induced by stimulation with the T cells are naïve T cells and/or memory T cells

5. The method according to claim
wherein
the T cells are memory T cells.

6. The method according to claim 1,
wherein
the T cells are of pro-inflammatory Th1 and/or Th17 cells.

7. The method according to claim
wherein
the Notch molecules are Notch receptors and/or Notch ligands.

8. The method according to claim 7,
wherein
the Notch receptors are Notch 1, 2, 3 and/or 4 and the Notch ligands are selected from the group comprising the Jagged family, in particular Jagged 1 and/or 2, and/or the Delta-like (DII) family, in particular DII1, 2, and/or 4.

9. The method according to claim 7,
wherein
Notch or Notch ligands and/or against STAT4-activating cytokines, especially IL-12, IL-23, IL-27 and/or their receptors, and/or by recombinant proteins or protein fragments or peptides of Notch or Notch ligands.

18. The method according to claim 1,
wherein
inhibition of STAT4 activation is effected by substances which inhibit the natural activation or activity of STAT4.

19. The method according to claim 1,
wherein
inhibition of Notch activation is effected by using pharmacological inhibitors of Notch activation, e.g. so-called γ-secretase inhibitors.

20. The method according to claim 1,
wherein
inhibition proceeds in the course of an active immune reaction, especially during infections, tumor diseases, vaccinations.

21. The method according to claim 1,
wherein
inhibition proceeds in the course of a vaccination using antigens in particular of tumors or pathogens together with adjuvants, preferably ligands for Toll-like receptors (TLR), anti-CD40 and/or CD40 ligand.

22. The method according to claim 21,
wherein
the adjuvants are derived from the group of ligands for TLR9, preferably oligonucleotides comprising methylated CpG motifs.

23. The method according to claim 21,
wherein
the adjuvants are derived from the group of antibodies and/or ligands for CD40.

24. The method according to claim 20,
wherein
inhibition proceeds in the course of a vaccination with dendritic cells or other antigen-presenting cells, in particular also by using dendritic cells or other antigen-presenting cells for immunization, wherein expression of Notch ligands is inhibited.

25. The method according to claim 24, wherein
expression of Notch ligands of the Delta-like family, especially of the ligands Delta-like 1 and Delta-like 4, is inhibited in the dendritic cells or other antigen-presenting cells.

26. The method according t claim 1, wherein the method inhibits inflammations.

27. The method according to claim 1, wherein the method provides immunosuppression.

28. The method according to claim 1, wherein the method induces interleukin 10 (IL-10).

29. The method according to claim 3, wherein the method activates an immune response.

30. The method according to claim 3, wherein the method provides vaccination enhancement.

31. The method according to claim 3, wherein the method inhibits interleukin 10 (IL-10) expression.

32. A production process for a medicament
comprising the method of claim 1, wherein the medicament induces interleukin 10 (IL-10) for the immunosuppression in organ transplantations.

33. The method according to claim 24, wherein the expression of Notch ligands is inhibited—by transduction with siRNA or antisense oligonucleotides against the Notch ligands.
